(19) United States
(12) Patent Application Publication
(10) Pub. No.: US 2025/0026716 A1
(43) Pub. Date: Jan. 23, 2025

(54) SYNTHESIS PROCESS OF FATTY ACYL TAURINE SALT

(71) Applicant: ZHANGJIAGANG GREAT CHEMICAL CO., LTD., Suzhou, Jiangsu (CN)

(72) Inventors: Yongqian HUANG, Suzhou, Jiangsu (CN); Youjiang XU, Suzhou, Jiangsu (CN); Hongjun ZHU, Suzhou, Jiangsu (CN)

(73) Assignee: ZHANGJIAGANG GREAT CHEMICAL CO., LTD., Suzhou, Jiangsu (CN)

(21) Appl. No.: 18/711,871

(22) PCT Filed: Nov. 21, 2022
(86) PCT No.: PCT/CN2022/133240

§ 371 Date: May 20, 2024

(30) Foreign Application Priority Data
Nov. 25, 2021 (CN) 202111415592X

(51) Int. Cl. C07C 303/32; C07C 51/41

(52) U.S. Cl. CPC C07C 303/32; C07C 51/412

(57) ABSTRACT

The present disclosure herein provides a synthesis process of fatty acyl taurine salt, belonging to the field of organic chemical synthesis technology. The synthesis process, comprising: adding fatty acid or ester thereof, taurine, and solvent into a reaction vessel, stirring well, and then adding a catalyst, heating the reaction under stirring conditions, and removing the generated water during the reaction, after the reaction being completed, the fatty acyl taurine salt product is obtained. The present disclosure directly uses taurine and fatty acid or ester thereof as raw materials for reaction, and by controlling the type of the catalyst and solvent as well as the reaction temperature, the yield of the product can be significantly improved, and the obtained fatty acyl taurine salt have high purity, low irritation, light color, and strong foaming ability.

### TECHNICAL FIELD

The present disclosure belongs to the field of organic chemical synthesis technology and specifically relates to a synthesis process of fatty acyl taurine salt.

### BACKGROUND

Fatty acyl taurine salt is a type of surfactant, the conventional production process involves first reacting fatty acid with acylation reagents (such as phosgene or phosphorus trichloride) to synthesize fatty acyl chloride, then, condensing fatty acyl chloride and amino acids under alkaline conditions to synthesize fatty acyl taurine salt, wherein the byproduct is sodium chloride, which requires acidification layering or crystallization separation to obtain high-quality products; the entire synthesis process is long, and the byproduct sodium chloride can cause unexpected effects under a large number of application conditions, such as causing low-temperature turbidity in the application formula.

In view of this, many patents have also attempted to use other methods to synthesize salt free fatty acyl taurine salt series surfactants. However, it is worth noting that most of the N-alkyloyl taurine salt or N-acyl taurine salt mentioned in previous patents are actually N-acylmethyltaurine salt.

The applicant found during the implementation process that due to the fact that methyl taurine salt is a secondary amine and taurine is a primary amine, the steric hindrance is smaller and easier to react, resulting in a better conversion rate.

The applicant also found during the implementation process that methyl taurine salt is prone to the following decomposition reactions at high temperatures:

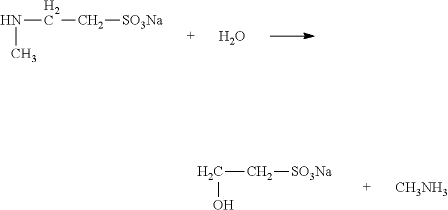

The methylamine in the decomposition product will further undergo acylation reaction with fatty acid:

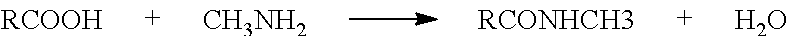

Due to the strong irritancy of methylamine or fatty acylmethylamine, the products obtained from the reaction of fatty acid or fatty acid ester with methyltaurine/methyltaurine salt will contain byproducts, whereby the products often need to undergo secondary purification and can be used in commercial products.

Until after 2000, with the rapid increase in the use of taurine as a food additive, taurine began industrial production and reached a production capacity of hundreds of thousands of tons, whereby the price of taurine greatly decreased, giving it more advantages compared to methyltaurine sodium as a raw material; it was precisely because this applicant discovered this trend that they first systematically attempted to use taurine to develop and synthesize fatty acyl taurine salt.

The applicant was pleasantly surprised to find that using taurine instead of methyl taurine has the following significant advantages:
however, due to its small steric hindrance, taurine salt have better selectivity in amidation reaction; under optimal reaction conditions, taurine does not decompose, resulting in higher yields, purity, less irritation, and milder products.

Day's U.S. Pat. No. 5,496,959 involves the preparation of N-acyl taurine salt by reacting carboxylic acid with “taurine salt” derivatives (defined as substituted 2-aminoalkane sulfonic acid and alkali metal salt thereof). In fact, all examples involve N-methyltaurine sodium, without involving taurine salt, moreover, with the implementation of the process in its examples, the conversion rate of the product is not high, there are many impurities, dark colors, and strong odors, making it difficult to truly have commercial value.

Burnett's U.S. Pat. No. 2,880,219 also teaches the preparation of N-acyl taurine salt from fatty acid and taurine, which are actually implemented using N-acyl methyl taurine salt. Excessive amounts of fatty acid are used to reduce the viscosity during the reaction process and reduce the decomposition of methyl taurine salt; but in reality, there has not been much substantial improvement, and excessive fatty acid will increase separation costs, whereby the true commercialization value has not been achieved.

Schenck et al.'s U.S. Pat. No. 3,232,968 disclosed a method for preparing N-acyl taurine salt using hypophosphorous acid. In reality, all examples involve N-methyltaurine sodium and do not involve taurine salt. Although hypophosphorous acid is used as an antioxidant, the final product color can only reach a minimum APHA of 2.5% aqueous solution to 10; in reality, concentrated solutions or solids still have darker colors, and the problem of multiple byproducts has not been solved.

Walele et al.'s U.S. Pat. No. 5,434,276 discloses a method for preparing acyl taurine salt, alkali metal borohydride and taurine salt (actually N-methyltaurine sodium) are first pretreated and heated, and then fatty acid preheated to reaction temperature are added. All examples use N-methyltaurine sodium, which also fails to avoid the problem of decomposition byproducts.

Chinese patent CN111902395A increases the content of N-methyltaurine salt to over 75%, thereby increasing the yield of alkyltaurine amide and reducing its browning risk. However, the application clearly records the alkali metal salt of fatty acid and N-methyltaurine, which still fails to solve the problem of N-methyltaurine decomposition.

Chinese patent application CN201510568940.5 discloses a synthesis method for lauroyl-sodium methyl taurate, which explicitly uses methyltaurine sodium as a solvent and liquid paraffin as a solvent; later, it is separated by extraction with water and cyclohexane, resulting in a long process step and significant emissions of three wastes.

There are also some prior arts that use taurine salt to produce N-acyl taurine, such as JP 2002-234868, which records a method for preparing acyl taurine salt by reacting fatty acid with taurine. From the examples, it can be seen that excessive fatty acid are still used to reduce viscosity; excessive fatty acid will increase separation costs or make it difficult to directly use in commercial products.

Chinese patent CN103857653A uses fatty acid ester and taurine salt to produce N-acylmethyltaurine sodium, avoiding the problem of decomposition of methyltaurine. However, the reaction temperature used is low, the catalyst efficiency is not strong enough, and the residual fatty acid ester content is high.

After long-term research, the applicant found that there is currently no industrialized supplier of N-acyl taurine salt surfactants in the world, and only reagent grade samples can be purchased. Similarly, N-acyl taurine salt surfactants have not appeared in the existing chemical substance list IECSC in China and various versions of the Used Cosmetic Raw Materials Catalogue in China.

Therefore, it is necessary to develop a synthesis process for fatty acyl taurine salt that directly use taurine and fatty acid or ester thereof as raw materials, with high process yield, light product color, few byproducts, and low irritation.

### SUMMARY

Based on the existing problems in the technology, the present disclosure aims to provide a synthesis process of fatty acyl taurine salt, which uses taurine and fatty acid or ester thereof as raw materials for reaction; by controlling the type of catalyst and solvent and reaction temperature, the yield of the product can be significantly improved, and the obtained fatty acyl taurine salt has high purity, low irritation, strong foaming ability, and light color.

The present disclosure is implemented through the following technical scheme.

A synthesis process of fatty acyl taurine salt, comprising:
adding fatty acid or ester thereof, taurine, and solvent into a reaction vessel, stirring well, and then adding a pH regulator, heating the reaction under stirring conditions, and removing the generated water during the reaction, after the reaction being completed, the fatty acyl taurine salt product is obtained.

Wherein, the fatty acyl taurine salt product is, but is not limited to, a solvate, hydrate, or pure fatty acyl taurine product of the fatty acyl taurine salt product.

In other preferred embodiments, the synthesis process needs to be carried out under the protection of nitrogen.

Wherein the fatty acid is C8-C22 fatty acid, including branched chain fatty acid and/or branched chain fatty acid;
preferably, the C8-C22 fatty acid is selected from one or more of lauric acid, coconut oil acid, octanoic acid, decanoic acid, myristic acid, palmitic acid, stearic acid, oleic acid, linoleic acid, arachidic acid, decanoic acid, and isostearic acid;
preferably, the C8-C22 fatty acid is selected from one or more of coconut oil acid, lauric acid, myristic acid, and stearic acid;
wherein the fatty acid ester is C1-C4 alcohol fatty acid ester. Preferably, the C1-C4 alcohol fatty acid ester includes but is not limited to one or more of methyl ester, ethyl ester, propylene glycol ester, glycerol ester, and isopropanol ester.

The solvent is a polyol solvent;
Preferably, the polyol solvent is C2-C10 high boiling point polyol;
further preferably, the C2-C10 high boiling point polyol is selected from one or more of glycerol, propylene glycol, ethylene glycol, erythritol, xylitol, pentanediol, hexanediol, and butanediol;
more preferably, the C2-C10 high boiling point polyol is selected from propylene glycol or/and glycerol;
most preferably, the C2-C10 high boiling point polyol is propylene glycol.

The equivalence ratio of the pH regulator to taurine is 0.9-1.2:1, preferably 0.93-1:1.

The pH regulator is an alkaline pH regulator; the alkaline pH regulator includes but is not limited to one or more of sodium hydroxide, potassium hydroxide, lithium hydroxide, magnesium hydroxide, calcium hydroxide, sodium carbonate, potassium carbonate, sodium bicarbonate, potassium bicarbonate, lithium carbonate, calcium oxide, sodium hydroxide, zinc oxide, sodium ethoxide, sodium methoxide, potassium ethoxide, potassium methoxide, triethanolamine, and triethylamine.

Preferably, the alkaline pH regulator is selected from one or more of sodium hydroxide, potassium hydroxide, sodium carbonate, and potassium carbonate.

The mass ratio of the taurine to the polyol is 1:0.3-6;
preferably, the mass ratio of the taurine to the polyol is 1:0.5-2;
further preferably, the mass ratio of the taurine to the polyol is 1:0.5-1.

The heating reaction temperature is 150° C.-220° C.;
preferably, the heating reaction temperature is 180° C.-210° C.

In some preferred embodiments, in the synthesis process, during the synthesis process, an alkaline metal salt catalyst and an auxiliary catalyst need to be added to the reaction system;
the mass ratio of the alkaline metal salt catalyst to the auxiliary catalyst is 0.5-6:1, preferably 1-3:1.

Wherein the alkaline metal salt catalyst is selected from one or more of sodium tert butanol, potassium tert butanol, sodium methanol, sodium ethanol, sodium isopropanol, potassium methanol, potassium ethanol, potassium isopropanol, and calcium oxide.

Preferably, the alkaline metal salt is selected from one or more of sodium tert butanol, sodium methoxide, and sodium ethanol.

The auxiliary catalyst is selected from one or more of sodium hypophosphate, sodium borohydride, zinc oxide, copper sulfate, sodium phosphite, hypophosphite, phosphite, boric acid, and phenylboronic acid.

Preferably, the auxiliary catalyst is selected from sodium hypophosphate or sodium borohydride.

During the implementation process, it was unexpectedly discovered that compared to conventional catalyst such as sodium hypophosphate or sodium phosphite, alkaline metal salt as catalyst can most effectively improve the conversion rate. At the same time, it was found that adding a mixed catalytic system to the reaction system is more conducive to the progress of the reaction; in the implementation process of the present disclosure, controlling the mass ratio of alkaline metal salt catalyst and auxiliary catalyst can significantly improve the conversion rate of the reaction, reduce the color and odor of the product, and reduce the dosage of catalyst added and the steps of post-treatment.

Compared with prior art, the beneficial effects of the present disclosure are:
Consumers are most concerned about the safety of personal care products, and as the main surface activity, its irritancy is largely related to impurity content, in addition to being limited by the main component itself. For example, in acyl amino acid salt synthesized by acyl chloride method, impurities in acyl chloride synthesis, residual acyl chloride, residual phosgene, etc., although in trace amounts, have a significant impact on irritancy. Alternatively, in the case of sodium methylcocoyl taurine directly synthesized from fatty acid and methyltaurine sodium, which contains a large amount of fatty acyl methylamine, the irritancy is significantly increased.

Personal care products are products that bring beauty to consumers, and their color and odor are important sensory needs. They are colorless and odorless, making consumers feel that the product is pure and safe.

The present disclosure directly uses taurine and fatty acid or ester thereof as raw materials for reaction; by controlling the molar ratio of taurine and fatty acid or ester thereof, catalyst, pH regulator, and solvent type, as well as the dosage and reaction temperature, the yield can be significantly improved, and the obtained fatty acyl taurine salt has high purity, low irritation, strong foaming ability, and light color.

### BRIEF DESCRIPTION OF THE DRAWINGS

FIG. 1 shows the infrared spectrum of sodium lauroyl taurine potassium bromide compressed tablet prepared in Example 1;

FIG. 2 shows the infrared spectrum of sodium lauroyl taurine potassium bromide compressed tablet prepared in Comparative Example 1;

FIG. 3 shows the GC plot of fatty acyl methyl taurine salt prepared in Comparative Example 2;

FIG. 4 shows the GC-MS qualitative analysis of lauroyl methylamine in fatty acyl methyl taurine salt prepared in Comparative Example 2

Figure marker: A represents the mass spectrum of sample RT=16.189 min; B represents the standard mass spectrum of lauroyl methylamine

### DETAILED DESCRIPTION OF THE EMBODIMENTS

The present disclosure will be further elaborated in detail by combining specific examples; the following examples are not intended to limit the present disclosure, but only to illustrate the present disclosure. Unless otherwise specified, the experimental methods used in the following examples, which do not specify specific conditions, are usually obtained through commercial channels under conventional conditions.

The following examples will further elaborate on the present disclosure, but these examples do not constitute any limitation on the present disclosure.

1. The purchasing method and model
of the reagents used in the example

| Raw material | Model | Manufacturer |
|---|---|---|
| Lauric acid | 1299 | Taiko Palm-Oleo |
| Coconut oleic acid | B1210 | (Zhangjiagang) |
| Myristic acid | 1499 | |
| Stearic acid | 1865 | |
| Glycerol | — | |
| Taurine | — | HUANGGANG |
| 40% N-methyltaurine sodium | — | YONGAN |
| aqueous solution | | Pharmaceutical |
| Propylene glycol | — | Shell |
| 1,2-propanediol | — | |
| Butanediol | — | Celanese |
| 1,3-Butanediol | — | |
| Erythritol | — | Shandong Sanyuan |
| Sodium hydroxide | — | Jiangsu Lee & Man |
| 32% sodium hydroxide aqueous | — | |
| solution | | |
| Anhydrous sodium carbonate | — | Jiangsu DEBANG |
| 90% potassium hydroxide | — | Zhenjiang YNID |
| Liquid paraffin | 26 # White | Shanghai Better |
| | oil | Chemical |
| Zinc oxide | — | National |
| Sodium hypophosphate | | Pharmaceutical |
| Magnesium sulfate acetic acid | — | Group |
| Sodium borohydride | — | |
| Calcium oxide | — | |
| Sodium chloride | — | |
| Trisodium phosphate | — | |
| Methyl laurate | — | |
| Laurylamide | — | TCI reagent |
| Lauroyl methylamine | — | |

The test method used in the examples of the present disclosure

| | Test method |
|---|---|
| 1. Component test in | |
| products | |
| Fatty acyl taurine salt | Using the bromocresol green method and two-phase |
| | titration |
| Fatty acid | Using the petroleum ether extraction method |
| Taurine | Using the carbon disulfide method |
| Methyltaurine sodium | Using the carbon disulfide method |
| Fatty acyl methylamine | Using the liquid phase external standard method |
| Fatty amides | Using the liquid phase external standard method |
| Other | Using the subtraction method |
| 2. Performance test | |
| Stimulus test | Using chicken embryo chorioallantoic membrane test |
| Color | Using instrumental analysis |
| Foam volume | Mixing method: take 2 g of sample, adding 150 mg/kg |
| | of hard water to 500 mL, and quickly stirring for 30 s |
| | at 1000 rpm. Record the amount of foam initially |
| | generated and the amount of foam 5 minutes later |

3. The yield in the following examples is equal to the molar amount of fatty acyl taurine salt or fatty acyl methyl taurine salt produced/the average molar amount of fatty acid and taurine input.

### Example 1 A Synthesis Process of Fatty Acyl Taurine Salt

comprising:
adding 200.32 g (1 mol) of lauric acid, 125.15 g (1 mol) of taurine, and 62.6 g of propylene glycol into a 1000 mL three-necked flask with stirring, stirring and mixing well, and then adding 38.2 g of sodium hydroxide; introducing nitrogen, keeping nitrogen protection during the reaction, heating to reaction temperature under stirring, removing water generated during the reaction, keeping the temperature until the reaction being completed, removing solvent and recovering, discharging to obtain 335.41 g of white solid, wherein the content of sodium lauroyl taurine is 87.3%, and the yield is 88.5%.

### Comparative Example 1 A Synthesis Process of Fatty Acyl Taurine Salt

comprising:
adding 200.32 g (1 mol) of lauric acid and 1.5 g (0.02 mol) of N, N-dimethylformamide into a 500 mL four-necked flask equipped with a thermometer, reflux condenser tube, gas guide tube, and stirring; raising the temperature to 60° C., and controlling the reaction temperature to 75° C.; within 8 hours, introducing 110 g (1.11 mol) of phosgene; the reaction being completed, and vacuum distillation was carried out to collect the 140-160° C. (1333 Pa) fraction, obtaining 196.5 g of lauryl chloride, and the yield is 90%;
(2) adding 264 g of water, 118.3 g of taurine, and 118.3 g of sodium hydroxide to the reaction vessel, stirring to dissolve, and preparing a 33% sodium taurine solution, then adding 132 g of acetone, stirring well, and cooling to below 10° C., slowly and uniformly adding 196.5 g of lauroyl chloride prepared in step (1), while dropping 118.3 g of 32% sodium hydroxide aqueous solution, controlling the pH of the reaction solution to be between 9-10, and continuing to react at 25° C. for 2 hours; the pH should still be maintained between 9-10 at the end, and the white paste like reactant lefted in the refrigerator overnight, filtered dry, and treated with ethanol before drying to obtain a white powder of 258.6 g, wherein the content of sodium lauroyl taurine is 93.2%.

The difference between Example 1 and Comparative Example 1 is that Example 1 adopts a one-step synthesis method to synthesize fatty acyl taurine salt (i.e. the method claimed for protection in the present disclosure), compared to the two-step synthesis method to synthesize fatty acyl taurine salt (i.e. the commonly used method in the prior art) in Document 1, the two methods are compared, please refer to Table 1 for details:

TABLE 1

| | Example 1 | Comparative Example 1 |
|---|---|---|
| Preventing waste | 0.006 | 2.154 |
| *Waste/Product | | |
| Low toxicity | No toxic chemicals | Phosgene, acetone, phosphorus |
| chemical | throughout the | trichloride, |
| synthesis | process | N,N-dimethylformamide: toxic |
| | | Fatty acid acyl chloride: strong |
| | | corrosiveness |
| Derivative | Only water is | Carbon dioxide, hydrogen |
| | produced | chloride, sodium chloride, water |
| Intrinsic safety | Can achieve no | Phosgene and phosphorus |
| chemistry for | hazardous chemicals | trichloride: toxicity |
| accident | throughout the entire | Phosphorus trichloride, acetone, |
| prevention | process | ethanol: flammable and explosive |

According to the test data in Table 1 above, it can be seen that, the preparation method of Example 1 is environmentally friendly, in line with the principles of green chemistry, and is a new generation of innovative green processes with no waste generated. The preparation method of Comparative Example 1 will generate a large amount of waste, such as acyl chloride evaporation residue, carbon dioxide, hydrogen chloride, crystallization separation wastewater (containing sodium chloride, fatty acid, amino acids), etc., which will produce toxic or flammable and explosive waste.

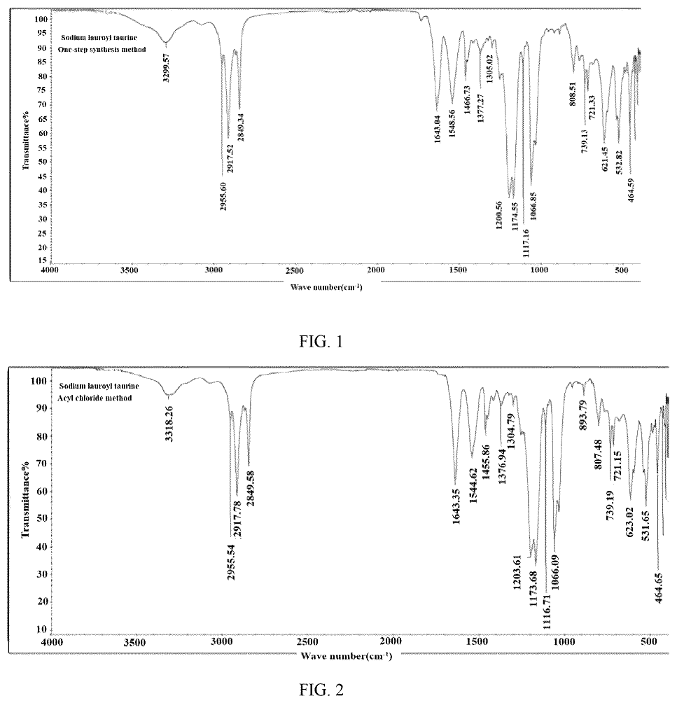

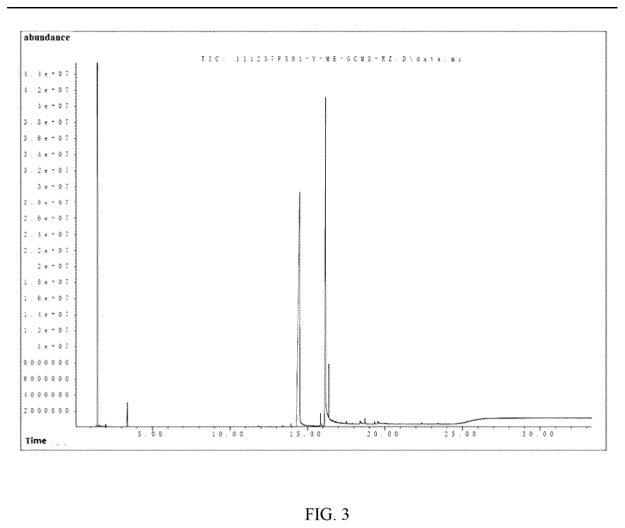

At the same time, in order to further confirm that the product prepared in Example 1 is sodium lauroyl taurine, infrared spectra of Example 1 and the product in Comparative Example 1 were performed, as shown in FIG. 1 and FIG. 2, wherein the fingerprint peaks of the two products completely match, proving that the two products are consistent.

### Examples 2-3 A Synthesis Process of Fatty Acyl Taurine Salt

comprising:
adding fatty acid, taurine, and solvent into a 1000 mL three-necked flask with stirring, stirring and mixing well, and then adding pH regulator and catalyst; introducing nitrogen, keeping nitrogen protection during the reaction, heating to reaction temperature under stirring, removing water generated during the reaction, keeping the temperature until the reaction being completed, removing solvent and recovering, discharging. The specific amount of addition is shown in Table 2.

TABLE 2

| Raw material components | | Example 2 | | Example 3 | |
|---|---|---|---|---|---|
| taurine | | 137.67 | g (1.1 mol) | 125.15 | g (1 mol) |
| Lauric acid | | 200.32 | g (1 mol) | 220.35 | g (1.1 mol) |
| Solvent | Propylene glycol | 96.4 | g | — | |
| | Butanediol | — | | 50.1 | g |
| PH regulator | sodium hydroxide | 44 | g | 40 | g |
| Alkaline metal | Sodium methoxide | 1.38 | g | 1.25 | g |
| salt catalyst | | | | | |
| Auxiliary | Sodium | 1.38 | g | 1.25 | g |
| catalyst | hypophosphate | | | | |

The reaction conditions of Example 2 are as follows: reacting at 180° C. for 6 hours to obtain 354.15 g of white solid product, wherein the content of sodium lauroyl taurine is 89.75%, and the yield is 91.88%.

The reaction conditions of Example 3 are as follows: reacting at 200° C. for 6 hours to obtain 367.5 g of white solid product, wherein the content of sodium cocoyl taurine is 89.35%, and the yield is 92.19%.

### Comparative Examples 2-3 A Synthesis Process of Fatty Acyl Taurine Salt

comprising:
adding fatty acid, methyltaurine sodium, and solvent into a 1000 mL three-necked flask with stirring, stirring and mixing well, and then adding pH regulator and catalyst; introducing nitrogen, keeping nitrogen protection during the reaction, heating to reaction temperature under stirring, removing water generated during the reaction, keeping the temperature until the reaction being completed, removing solvent and recovering, discharging. The specific amount of addition is shown in Table 3.

TABLE 3

| | | Comparative | | Comparative | |
|---|---|---|---|---|---|
| Raw material components | | Example 2 | | Example 3 | |
| Methyltaurine sodium (40%) | | 443.18 | g (1.1 mol) | 402.9 | g (1 mol) |
| Lauric acid | | 200.32 | g (1 mol) | 220.35 | g (1.1 mol) |
| Solvent | Propylene glycol | 124.1 | g | — | |
| | Butanediol | — | | 64.5 | g |
| PH regulator | Sodium hydroxide | | | 40 | g |
| Alkaline metal | Sodium methoxide | 1.77 | g | 1.61 | g |
| salt catalyst | | | | | |
| Auxiliary | Sodium | 1.77 | g | 1.61 | g |
| catalyst | hypophosphate | | | | |

The difference between Comparative Example 2 and Example 2 is that methyltaurine sodium is used instead of taurine and pH regulator to obtain 377.67 g of burnt yellow solid product, wherein lauroyl-sodium methyl taurate is 66.53%, and the yield is 69.67%.

The difference between Comparative Example 3 and Example 3 is that methyltaurine sodium is used instead of taurine and pH regulator to obtain 394.5 g of burnt yellow solid product, wherein lauroyl-sodium methyl taurate is 65.48%, and the yield is 69.71%.

Perform yield and content test on products prepared in Examples 2-3 and Comparative Examples 2-3, as shown in Table 4.

TABLE 4

| | Example | Example | Comparative | Comparative |
|---|---|---|---|---|
| | 2 | 3 | Example 2 | Example 3 |
| Product | 89.75% | 89.35% | 66.53% | 65.48% |
| content | | | | |
| Yield | 91.88% | 92.19% | 69.67% | 69.71% |

Perform irritancy test on products prepared in Examples 2-3 and Comparative Examples 2-3, using the chicken embryo chorioallantoic membrane method, as shown in Table 5.

TABLE 5

| | Example | Example | Comparative | Comparative |
|---|---|---|---|---|
| | 2 | 3 | Example 2 | Example 3 |
| Bleeding time | 41.5 | 39.5 | 9.8 | 8.5 |
| Hemolysis | 301 | 301 | 116.5 | 136.2 |
| time | | | | |
| Coagulation | 301 | 301 | 301 | 301 |
| time | | | | |
| IS value | 4.33 | 4.36 | 9.16 | 8.72 |

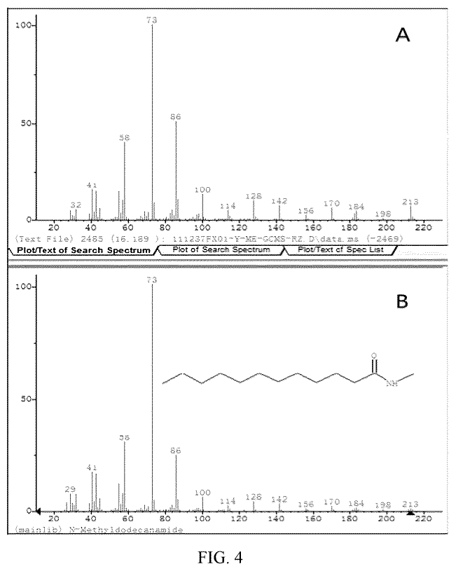

After testing, it can be seen that the product obtained from the Comparative Examples 2-3 has higher irritancy compared to the product obtained from the Examples 2-3; to further explore the reason for the strong irritancy of the product obtained from the Comparative Examples 2-3, GC-MS analysis was performed on the product obtained from the Comparative Example 2, as shown in FIGS. 3 and 4. The product was tested using the liquid phase external standard method, and the test results are shown in Table 6.

TABLE 6

| | Example | Example | Comparative | Comparative |
|---|---|---|---|---|
| | 2 | 3 | Example 2 | Example 3 |
| Lauramide content | — | — | — | — |
| Lauryl methylamine | — | — | 10.2% | 7.22% |
| content | | | | |

From the test results in Table 6 above, it can be seen that the products on Comparative Examples 2-3 contain 10.2% and 7.22% of lauroyl methylamine, respectively; lauroyl methylamine is a highly permeable and highly polar amine with strong irritancy; while the product obtained from Examples 2-3 does not contain laurylamide, therefore, it can be concluded that even if fatty acyl taurine salt is synthesized using a one-step method, the product obtained using methyltaurine sodium as the raw material has strong irritancy.

The use of taurine instead of methyl taurine salt to synthesize fatty acyl taurine salt has the advantages of high yield, high product purity, and low irritation.

### Examples 4-5 A Synthesis Process of Fatty Acyl Taurine Salt

comprising:
adding fatty acid, taurine, and solvent into a 1000 mL three-necked flask with stirring, stirring and mixing well, and then adding pH regulator and catalyst;
introducing nitrogen, keeping nitrogen protection during the reaction, heating to reaction temperature under stirring, removing water generated during the reaction, keeping the temperature until the reaction being completed, removing solvent and recovering, discharging. The specific amount of addition is shown in Table 7.

TABLE 7

| Raw material components | | Example 4 | | Example 5 | |
|---|---|---|---|---|---|
| Taurine | | 125.15 | g (1 mol) | 125.15 | g (1 mol) |
| Coconut oleic acid | | 210 | g (1 mol) | 210 | g (1 mol) |
| Solvent | Propylene glycol | 37.55 | g | 250.3 | g |
| PH regulator | Sodium carbonate | 63 | g | 63 | g |
| Alkaline metal | Sodium methoxide | 1.25 | g | 1.25 | g |
| salt catalyst | | | | | |
| Auxiliary | Sodium | 1.25 | g | 1.25 | g |
| catalyst | borohydride | | | | |

The reaction conditions of Example 4 are as follows: the mass ratio of taurine to propylene glycol is 1:0.3, and the reaction is carried out at 180° C. for 6 hours to obtain 345.03 g of white solid, wherein the content of sodium cocoyl taurine is 95.74%, and the yield is 97.4%. After testing, the color of the 30% aqueous solution is 13 Hazen.

The reaction conditions of Example 5 are as follows: the mass ratio of taurine to solvent propylene glycol is 1:2, and the reaction is carried out at 180° C. for 6 hours to obtain 366.39 g of white solid, wherein the content of sodium cocoyl taurine is 89.42%, and the yield is 96.6%. After testing, the color of the 30% aqueous solution is 17 Hazen.

### Comparative Examples 4-5 A Synthesis Process of Fatty Acyl Taurine Salt

comprising:
adding fatty acid, taurine, and solvent into a 1000 mL three-necked flask with stirring, stirring and mixing well, and then adding pH regulator and catalyst; introducing nitrogen, keeping nitrogen protection during the reaction, heating to reaction temperature under stirring, removing water generated during the reaction, keeping the temperature until the reaction being completed, removing solvent and recovering, discharging. The specific amount of addition is shown in Table 8.

TABLE 8

| | | Comparative | | Comparative | |
|---|---|---|---|---|---|
| Raw material components | | Example 4 | | Example 5 | |
| Taurine | | 125.15 | g (1 mol) | 125.15 | g (1 mol) |
| Coconut oleic acid | | 210 | g (1 mol) | 210 | g (1 mol) |
| Solvent | Propylene glycol | — | | 876.05 | g |
| PH regulator | Sodium carbonate | 63 | g | 63 | g |
| Alkaline metal | Sodium methoxide | 1.25 | g | 1.25 | g |
| salt catalyst | | | | | |
| Auxiliary | Sodium borohydride | 1.25 | g | 1.25 | g |
| catalyst | | | | | |

The difference between Comparative Example 4 and Example 4 is that if no solvent is used in the reaction system, the raw material taurine cannot be dissolved, the reaction cannot proceed, and after heating for 2 hours, the material has become burnt black and odorous.

The difference between Comparative Example 5 and Example 4 is that the mass ratio of taurine to propylene glycol is 1:7, resulting in a white solid of 424.33 g, wherein the content of sodium cocoyl taurine is 60.1%, and the yield is 75.2%. After testing, the color of the 30% aqueous solution is 13 Hazen.

Through the above experiments, it can be seen that when there is no solvent or the solvent amount is insufficient, taurine cannot dissolve, resulting in difficult mass and heat transfer, and the reaction cannot proceed. However, when the solvent amount exceeds the limit, the ratio of esterification side reactions increases, the purity of the product decreases, and the final yield significantly decreases.

### Example 6 A Synthesis Process of Fatty Acyl Taurine Salt

comprising:
adding 300.3 g (1.1 mol) of stearic acid, 125.15 g (1 mol) of taurine, and 87.6 g of glycerol into a 1000 mL three-necked flask with stirring, stirring and mixing well, and then adding 40 g of sodium hydroxide, 1.88 g of sodium methoxide, and 0.63 g of sodium hypophosphate; introducing nitrogen, keeping nitrogen protection during the reaction, heating to 210° C. under stirring, removing water generated during the reaction, keeping the temperature until the reaction being completed, removing solvent and recovering. Finally, 447.59 g of white solid is obtained, wherein the content of sodium stearoyl taurine is 83.56%, and the yield is 88.57%.

Wherein the catalyst mass accounts for 2% of the mass of taurine, and the mass ratio of sodium methoxide to sodium hypophosphate is 3:1.

### Example 7 A Synthesis Process of Fatty Acyl Taurine Salt

The difference from Example 6 is that sodium tert butanol 0.63 g, sodium hypophosphate 0.63 g. The catalyst mass accounts for 1% of the mass of taurine, and the mass ratio of sodium tert butanol to sodium hypophosphate is 1:1. Finally, 447.46 g of white solid is obtained, wherein the content of sodium stearoyl taurine is 82.95%, and the yield is 87.9%.

### Comparative Example 6

The process difference from Example 6 is only that no catalyst was added. Finally, 443.78 g of white solid is obtained, wherein the content of sodium stearoyl taurine is 77.21%, and the yield is 81.14%.

### Comparative Example 7

The process difference from Example 6 is only that 2.5 g of sodium methoxide is added without any auxiliary catalyst. The catalyst mass accounts for 2% of the mass of taurine, Finally, 446.33 g of white solid is obtained, wherein the content of sodium stearoyl taurine is 80.22%, and the yield is 84.81%.

### Comparative Example 8

The process difference from Example 6 is only that without the addition of alkaline metal salt catalyst, sodium hypophosphate 2.5 g. The catalyst mass accounts for 2% of the mass of taurine, Finally, 447.3 g of white solid is obtained, wherein the content of sodium stearoyl taurine is 77.96%, and the yield is 82.48%.

### Comparative Example 9

The process difference from Example 6 is only that 0.63 g of sodium tert butanol and 1.88 g of sodium hypophosphate. The catalyst mass accounts for 2% of the mass of taurine, and the mass ratio of sodium tert butanol to sodium hypophosphate is 1:4. Finally, 445.54 g of white solid is obtained, wherein the content of sodium stearoyl taurine is 80.1%, and the yield is 84.76%.

### Comparative Example 10

The process difference from Example 6 is only that 2.09 g of sodium tert butanol and 0.41 g of sodium hypophosphate. The catalyst mass accounts for 2% of the mass of taurine, and the mass ratio of sodium tert butanol to sodium hypophosphate is 5:1. Finally, 447.62 g of white solid is obtained, wherein the content of sodium stearoyl taurine is 80.5% with a yield of 85.12%.

### Comparative Example 11

The process difference from Example 6 is only that 2.5 g of zinc oxide is used as the catalyst. The catalyst mass accounts for 2% of the mass of taurine. Finally, 448.62 g of white solid is obtained, wherein the content of sodium stearoyl taurine is 75.95%, and the yield is 80.29%.

According to the comparison between Example 6 and Comparative Examples 6-11 mentioned above, it can be seen that using zinc oxide as a catalyst for Comparative Example 11 results in a final yield equivalent to that without a catalyst, and the yield is about 80%. However, using alkaline metal salt as a catalyst can significantly increase the yield to 84.48%. Furthermore, when using an appropriate ratio of alkaline metal salt and auxiliary catalyst, the yield can be further increased to 88.57%. Furthermore, when using an appropriate ratio of alkaline metal salt and auxiliary catalyst, the yield can be increased to 87.9% by using 1% taurine mass.

In summary, alkaline metal salt is effective catalyst for catalyzing the one-step synthesis of fatty acyl taurine salt from taurine and fatty acid. Furthermore, a suitable ratio of alkaline metal salt combined with auxiliary catalyst can further improve yield or reduce catalyst dosage.

### Example 8 A Synthesis Process of Fatty Acyl Taurine Salt

comprising:
adding 210 g (1 mol) of coconut oil acid, 125.15 g (1 mol) of taurine, and 62.6 g of propylene glycol into a 1000 mL three-necked flask with stirring, stirring and mixing well, and then adding 53.3 g of potassium hydroxide, 1.9 g of sodium methoxide, and 1.9 g of sodium hypophosphate. Introducing nitrogen, keeping nitrogen protection during the reaction, heating to 150° cunder stirring, removing water generated during the reaction, keeping the temperature until the reaction being completed, removing solvent and recovering. Finally, 345.95 g of white solid is obtained, wherein the content of potassium cocoyl taurine is 90.24%, and the yield is 91.6%.

### Example 9

The process difference from Example 8 is only that the reaction temperature is 210° C. Finally, 346.39 g of white solid is obtained, wherein the content of potassium cocoyl taurine is 95.5%, and the yield is 97.3%.

### Comparative Example 12

The process difference from Example 8 is only that the reaction temperature is 120° C. The reaction temperature is too low and the reaction has not been carried out.

### Comparative Example 13

The process difference from Example 8 is only that the reaction temperature is 230° C. Finally, 345.04 g of burnt yellow solid is obtained, wherein the content of potassium cocoyl taurine is 89.96%, and the yield is 91.1%. Moreover, coconut oil amide is found in the product.

To further demonstrate the effectiveness of Examples 8-9, the applicant also tested the color of 30% aqueous solution of the product of Examples 8-9 and Comparative Example 13.

TABLE 9

| | Example | Example | Comparative | Comparative |
|---|---|---|---|---|
| | 8 | 9 | Example 12 | Example 13 |
| Reaction | 150 | 210 | 120 | 230 |
| temperature, ° C. | | | | |
| 30% aqueous | 7 Hazen | 25 Hazen | — | 103 Hazen |
| solution color | | | | |

According to the data in Table 9 above, it can be seen that when using a reaction temperature lower than 150° C. for Comparative Example 12, the reaction cannot proceed; when using a reaction temperature higher than 210° C. for Comparative Example 13, more impurities are generated, fatty amides are detected, and the product color is darker.

### Example 10 A Synthesis Process of Fatty Acyl Taurine Salt

comprising:
adding 228.37 g (1 mol) of myristic acid, 125.15 g (1 mol) of taurine, and 37.5 g of propylene glycol into a 1000 mL three-necked flask with stirring. stirring and mixing well, and then adding 37.2 g of potassium hydroxide, 1.25 g of sodium methoxide, and 1.25 g of sodium hypophosphate. Introducing nitrogen, keeping nitrogen protection during the reaction, heating to 190° cunder stirring, removing water generated during the reaction, keeping the temperature until the reaction being completed, removing solvent and recovering. Finally, 363.26 g of white solid is obtained, wherein the content of sodium myristoyl taurine is 93.74%, and the yield is 95.2%.

The equivalence ratio of sodium hydroxide to taurine equivalent is 0.93:1.

### Example 11

The process difference from Example 10 is only that the dosage of sodium hydroxide is 44 g, and the equivalence ratio to taurine is 1.1:1. Finally, 367.41 g of white solid is obtained, wherein the content of sodium myristoyl taurine is 93.5%, and the yield is 96.1%.

### Comparative Example 14

The process difference from Example 10 is only that the dosage of sodium hydroxide is 34 g, and the equivalence ratio to taurine is 0.85:1. Due to insufficient alkalinity, the acylation reaction is not complete. Finally, 361.67 g of light yellow solid is obtained, wherein the content of sodium myristoyl taurine is 83.23%, and the yield is 84.2%.

### Comparative Example 15

The process difference from Example 10 is only that the dosage of sodium hydroxide is 52 g, and the equivalence ratio to taurine is 1.3:1. Finally, 375.47 g of burnt yellow solid is obtained, wherein the content of sodium myristoyl taurine is 86.65%, and the yield is 91%, and myristamide content is found in the product, with a content of 0.89%.

Tests are conducted on products of Examples 10-11 and Comparative Examples 14-15, and the test results are shown in Table 10.

TABLE 10

| | Example | Example | Comparative | Comparative |
|---|---|---|---|---|
| | 10 | 11 | Example 14 | Example 15 |
| Sodium myristoyl | 93.74% | 93.5% | 83.23% | 86.65% |
| taurine | | | | |
| Yield | 95.2% | 96.1% | 84.2% | 91% |
| Myristamide | — | — | — | 0.89% |

According to the test data in Table 10 above, it can be seen that for the pH regulator and taurine equivalent ratio in Comparative Example 14, which is less than 0.9, the yield of sodium fatty acyl taurine is very low; however, for a pH regulator with a taurine equivalent ratio exceeding 1.2 in Comparative Example 15, it also leads to a decrease in the yield of sodium fatty acyl taurine and causes partial decomposition of taurine, resulting in the production of fatty amides and increased irritation. At the same time, excessive pH regulator remain in the product, making it highly alkaline and increasing the amount of pH regulator used in the application process.

### Example 12 A Synthesis Process of Fatty Acyl Taurine Salt

comprising:
adding 214.35 g (1 mol) of methyl laurate, 125.15 g (1 mol) of taurine, and 37.5 g of glycerol into a 1000 mL three-necked flask with stirring. stirring and mixing well, and then adding 37.2 g of sodium hydroxide, 1.25 g of sodium methoxide, and 1.25 g of sodium hypophosphate. Introducing nitrogen, keeping nitrogen protection during the reaction, heating to 190° C. under stirring, removing water generated during the reaction, keeping the temperature until the reaction being completed, removing solvent and recovering. Finally, 367.45 g of white solid is obtained, wherein the content of sodium lauroyl taurine is 95.94%, and the yield is 98.6%.

### Example 13 A Synthesis Process of Fatty Acyl Taurine Salt

The only difference from Example 12 is that methyl laurate is replaced with coconut oil 222.7 g (0.333 mol). Finally, 373.13 g of white solid is obtained, wherein the content of sodium cocoyl taurine is 91.31%, and the yield is 95.3%.

### Comparative Example 16 A Synthesis Process of Fatty Acyl Taurine Salt

comprising:
adding 214.35 g (1 mol) of methyl laurate, 125.15 g (1 mol) of taurine, and 37.5 g of glycerol into a 1000 mL three-necked flask with stirring, stirring and mixing well, and then adding 37.2 g of sodium hydroxide and 2.5 g of calcium oxide. Introducing nitrogen, keeping nitrogen protection during the reaction, heating to 140° C. under stirring, removing water generated during the reaction, keeping the temperature until the reaction being completed, removing solvent and recovering. Wherein the content of sodium lauroyl taurine is 86.83%, and the yield is 95.5%.

This synthesis method refers to the preparation method disclosed in the prior art (CN103857653A) for implementation.

### Comparative Example 17 A Synthesis Process of Fatty Acyl Taurine Salt

The only difference from Comparative Example 16 is that methyl laurate is replaced with coconut oil 222.7 g (0.333 mol), 366.94 g of white past is obtained, wherein the content of sodium cocoyl taurine is 83.89%, and the yield is 93.7%.

Tests are conducted on products of Examples 12-13 and Comparative Examples 16-17, and the results are shown in Table 11.

TABLE 11

| | Example | Example | Comparative | Comparative |
|---|---|---|---|---|
| | 12 | 13 | Example 16 | Example 17 |
| Sodium lauroyl | 95.94% | — | 91.83% | — |
| taurine | | | | |
| Sodium cocoyl | — | 91.31% | — | 88.89% |
| taurine | | | | |
| Methyl laurate | 0.59% | — | 3.02% | — |
| Coconut oil | — | 1.56% | — | 4.14% |
| Yield | 98.6% | 95.3% | 95.5% | 93.7% |

For further comparison, foaming performance tests are conducted on products of Examples 12-13 and Comparative Examples 16-17, and the test results are shown in Table 12.

TABLE 12

| | Example | Example | Comparative | Comparative |
|---|---|---|---|---|
| | 12 | 13 | Example 16 | Example 17 |
| Foam volume, | 1240 | 970 | 860 | 780 |
| mL | | | | |

According to the test data in Table 12 above, it can be seen that using calcium oxide as a catalyst for Comparative Example 16 and Comparative Example 17 has a relatively low process yield, and there are many fatty acid ester in the product, which reduces the foaming performance, while the present disclosure uses sodium methoxide and sodium hypophosphate as catalyst to improve the foaming performance of the product under the condition of ensuring product content.

The above shows and describes the basic principles, main features, and advantages of the present disclosure. A person skilled in the art should understand that the present disclosure is not limited by the above examples. The above examples and descriptions only explain the principles of the present disclosure; without departing from the spirit and scope of the present disclosure, there will be various changes and improvements in the present disclosure, all of which fall within the scope of the claimed protection. The scope of protection of the present disclosure is defined by the accompanying claims and their equivalents.

What is claimed is:

1. A synthesis process of fatty acyl taurine salt, comprising:
adding fatty acid or ester thereof, taurine, and solvent into a reaction vessel, stirring well, and then adding a pH regulator, heating the reaction under stirring conditions, and removing the generated water during the reaction, after the reaction being completed, the fatty acyl taurine salt product is obtained.

2. The synthesis process according to claim 1, wherein in the synthesis process, during the reaction process, a catalyst needs to be added to the reaction system, and the catalyst is an alkaline metal salt catalyst and/or auxiliary catalyst.

3. The synthesis process according to claim 1, wherein the fatty acid is C8-C22 fatty acid; the fatty acid ester is C1-C4 alcohol fatty acid ester.

4. The synthesis process according to claim 3, wherein the C8-C22 fatty acid is selected from one or more of lauric acid, coconut oil acid, octanoic acid, decanoic acid, myristic acid, palmitic acid, stearic acid, oleic acid, linoleic acid, and isostearic acid; the C1-C4 alcohol fatty acid ester is selected from one or more of methyl ester, ethyl ester, propylene glycol ester, glycerol ester, and isopropanol ester.

5. The synthesis process according to claim 4, wherein the C8-C22 fatty acid is coconut oil acid or lauric acid.

6. The synthesis process according to claim 1, wherein the solvent is a polyol solvent.

7. The synthesis process according to claim 6, wherein the polyol solvent is a C2-C10 high boiling point polyol.

8. The synthesis process according to claim 7, wherein the C2-C10 high boiling point polyol is selected from one or more of glycerol, propylene glycol, ethylene glycol, erythritol, xylitol, pentanediol, hexanediol, and butanediol.

9. The synthesis process according to claim 8, wherein the C2-C10 high boiling point polyol is selected from propylene glycol or/and glycerol.

10. The synthesis process according to claim 1, wherein the pH regulator is an alkaline pH regulator; the alkaline pH regulator is selected from but not limited to one or more of sodium hydroxide, potassium hydroxide, lithium hydroxide, magnesium hydroxide, calcium hydroxide, sodium carbonate, potassium carbonate, sodium bicarbonate, potassium bicarbonate, lithium carbonate, calcium oxide, sodium oxide, zinc oxide, sodium ethanol, sodium methoxide, potassium methoxide, potassium methoxide, triethanolamine, and triethylamine.

11. The synthesis process according to claim 10, wherein the alkaline pH regulator is one or more of sodium hydroxide, potassium hydroxide, sodium carbonate, and potassium carbonate.

12. The synthesis process according to claim 10, wherein the equivalence ratio of the alkaline pH regulator to taurine is 0.9-1.2:1.

13. The synthesis process according to claim 12, wherein the equivalence ratio of the alkaline pH regulator to taurine is 0.93-1.0:1.

14. The synthesis process according to claim 2, wherein the alkaline metal salt catalyst is selected from one or more of sodium tert butanol, potassium tert butanol, sodium methanol, sodium ethanol, sodium isopropanol, potassium methanol, potassium ethanol, potassium isopropanol, and calcium oxide.

15. The synthesis process according to claim 14, wherein the alkaline metal salt catalyst is selected from one or more of sodium tert butanol, sodium methanol, and sodium ethanol.

16. The synthesis process according to claim 2, wherein the auxiliary catalyst is selected from one or more of sodium hypophosphate, sodium borohydride, zinc oxide, copper sulfate, sodium phosphite, hypophosphite, phosphite, boric acid, and phenylboronic acid.

17. The synthesis process according to claim 16, wherein the auxiliary catalyst is sodium hypophosphate or sodium borohydride.

18. The synthesis process according to claim 2, wherein the mass ratio of the alkaline metal salt catalyst to the auxiliary catalyst is 0.5-6:1.

19. The synthesis process according to claim 18, wherein the mass ratio of the alkaline metal salt catalyst to the auxiliary catalyst is 1:3:1.

20. The synthesis process according to claim 1, wherein the mass ratio of the taurine to the polyol is 1:0.3-6.

21. The synthesis process according to claim 1, wherein the heating reaction temperature is 150° C.-220° C.

22. The synthesis process according to claim 21, wherein the heating reaction temperature is 180° C.-210° C.

23. The synthesis process according to claim 1, wherein the fatty acyl taurine salt product is, but is not limited to, a solvant, hydrate, or pure fatty acyl taurine product of the fatty acyl taurine salt product.
